SEQUENCE LISTING (18 sequences)

SEQ ID NO: 1 - protein, 5 aa, synthetic construct
GYNMN

SEQ ID NO: 2 - protein, 16 aa, synthetic construct
NIDPYYGDTDYDQKFK

SEQ ID NO: 3 - protein, 11 aa, synthetic construct
GAHRDYYAMDY

SEQ ID NO: 4 - protein, 11 aa, synthetic construct
RTSENIYIYLA

SEQ ID NO: 5 - protein, 7 aa, synthetic construct
NAKTLAE

SEQ ID NO: 6 - protein, 9 aa, synthetic construct
QQHYGTPYT

SEQ ID NO: 7 - protein, 120 aa, synthetic construct
EVHLQQSGPELEKPGASVKISCKASGYSFTGYNMNWVKQSNGKCLEWIGNIDPYYGDTDYDQKFKGKATLTADKSSNTVYMQLQSLTSEDSAVYYCARGAHRDYYAMDYWGQGTSVTVSS

SEQ ID NO: 8 - protein, 15 aa, synthetic construct
GGGGSGGGGSGGGGS

SEQ ID NO: 9 - protein, 107 aa, synthetic construct
DIQMTQSPASLSASVGETVTITCRTSENIYIYLAWYQQKQGKSPQLLVYNAKTLAEGVPSRFSGSGSGTQFSLKINGLQPEDFGGYYCQQHYGTPYTFGCGTKLEKK

SEQ ID NO: 10 - DNA, 726 bp, synthetic construct
  source 1..726: mol_type = other DNA
gaggtccacctgcagcagtctggacctgagctggagaagcctggcgcttcagtgaagatatcctgcaaggcttctggttactcattcactggctacaacatgaactgggtgaagcagagcaatggcaagtgccttgagtggattggaaatattgatccttactatggtgatactgactacgaccagaagttcaagggcaaggccacattgactgcagacaaatcctccaacacagtctacatgcagctccagagcctgacatctgaggactctgcagtctattactgtgcaagaggggcccatagggattactatgctatggactactggggtcaagggacctcagtcaccgtctcctcaggtggtggcggttcaggcggaggtggctctggcggtggcggatcggacatccagatgactcagtctccagcctccctatctgcatctgtgggagaaactgtcaccatcacatgtcgaacaagtgagaatatttacatttatttagcatggtatcagcagaaacagggaaaatctcctcagctcctggtctataatgcaaaaaccttagcagaaggtgtgccatcaaggttcagtggcagtgggtcaggcacacagttttctctgaagatcaacggcctgcagcctgaagattttgggggttattactgtcaacagcattatggcactccgtacacgttcggatgcgggaccaagctggaaaaaaaa

SEQ ID NO: 11 - protein, 22 aa, synthetic construct
MVLLVTSLLLCELPHPAFLLIP

SEQ ID NO: 12 - protein, 242 aa, synthetic construct
EVHLQQSGPELEKPGASVKISCKASGYSFTGYNMNWVKQSNGKSLEWIGNIDPYYGDTDYDQKFKGKATLTADKSSNTVYMQLQSLTSEDSAVYYCARGAHRDYYAMDYWGQGTSVTVSSGGGGSGGGGSGGGGSDIQMTQSPASLSASVGETVTITCRTSENIYIYLAWYQQKQGKSPQLLVYNAKTLAEGVPSRFSGSGSGTQFSLKINGLQPEDFGGYYCQQHYGTPYTFGGGTKLEKK

SEQ ID NO: 13 - protein, 83 aa, synthetic construct
FVPVFLPAKPTTTPAPRPPTPAPTIASQPLSLRPEACRPAAGGAVHTRGLDFACDIYIWAPLAGTCGVLLLSLVITLYCNHRN

SEQ ID NO: 14 - protein, 41 aa, synthetic construct
RSKRSRLLHSDYMNMTPRRPGPTRKHYQPYAPPRDFAAYRS

SEQ ID NO: 15 - protein, 47 aa, synthetic construct
RFSVVKRGRKKLLYIFKQPFMRPVQTTQEEDGCSCRFPEEEEGGCEL

SEQ ID NO: 16 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 549 aa, synthetic construct
MVLLVTSLLLCELPHPAFLLIPEVHLQQSGPELEKPGASVKISCKASGYSFTGYNMNWVKQSNGKSLEWIGNIDPYYGDTDYDQKFKGKATLTADKSSNTVYMQLQSLTSEDSAVYYCARGAHRDYYAMDYWGQGTSVTVSSGGGGSGGGGSGGGGSDIQMTQSPASLSASVGETVTITCRTSENIYIYLAWYQQKQGKSPQLLVYNAKTLAEGVPSRFSGSGSGTQFSLKINGLQPEDFGGYYCQQHYGTPYTFGGGTKLEKKAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 527 aa, synthetic construct
EVHLQQSGPELEKPGASVKISCKASGYSFTGYNMNWVKQSNGKSLEWIGNIDPYYGDTDYDQKFKGKATLTADKSSNTVYMQLQSLTSEDSAVYYCARGAHRDYYAMDYWGQGTSVTVSSGGGGSGGGGSGGGGSDIQMTQSPASLSASVGETVTITCRTSENIYIYLAWYQQKQGKSPQLLVYNAKTLAEGVPSRFSGSGSGTQFSLKINGLQPEDFGGYYCQQHYGTPYTFGGGTKLEKKAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2024/0293546 A1
(43) Pub. Date: Sep. 5, 2024

(54) D2C7 EGFR AND EGFR VIII BI-SPECIFIC CHIMERIC ANTIGEN RECEPTOR CONSTRUCTS AND METHODS OF MAKING AND USING SAME

(71) Applicant: Duke University, Durham, NC (US)

(72) Inventors: Peter FECCI, Durham, NC (US); Darell BIGNER, Durham, NC (US); Daniel LANDI, Durham, NC (US); Daniel WILKINSON, Durham, NC (US); Katherine RYAN, Durham, NC (US)

(21) Appl. No.: 18/574,656

(22) PCT Filed: Jul. 1, 2022
(86) PCT No.: PCT/US2022/073371

§ 371 Date: Dec. 27, 2023

Related U.S. Application Data
(60) Provisional application No. 63/217,399, filed on Jul. 1, 2021.

(51) Int. Cl. A61K 39/00; A61P 35/04; C07K 14/705; C07K 14/725; C07K 16/28; C12N 5/0783

(52) U.S. Cl. CPC A61K 39/464404; A61K 39/4611; A61K 39/4631; A61P 35/04; C07K 14/7051; C07K 14/70521; C07K 14/70578; C07K 16/2863; C12N 5/0636; C07K 2317/622; C07K 2319/02; C07K 2319/03; C12N 2510/00

(57) ABSTRACT

The present disclosure provides, in part, a novel chimeric antigen receptor (CAR) T cell that will simultaneously target wildtype EGFR (EGFRwt) and the vIII variant (EGFRvIII) and methods of making and using same.

### CROSS-REFERENCE TO RELATED APPLICATIONS

The present application claims priority to U.S. Provisional Patent Application No. 63/217,399, filed Jul. 1, 2021, the entire contents of which are hereby incorporated by reference.

### STATEMENT REGARDING FEDERALLY SPONSORED RESEARCH

N/A

### REFERENCE TO A SEQUENCE LISTING

The contents of the electronic sequence listing (15555400660.xml; Size: 19,015 bytes; and Date of Creation: Jun. 30, 2022) is herein incorporated by reference in its entirety.

### FIELD OF THE INVENTION

The disclosed technology is generally directed to therapeutic compositions and methods for the treatment of solid tumors. More particularly the technology is directed to the use of CAR-T cells in the treatment of solid tumors of the central nervous system.

### BACKGROUND OF THE INVENTION

According to the World Health Organization, cancer is responsible for close to 10 million deaths per year worldwide. There is a critical need to advance cancer treatments beyond their current state. Immune-based therapies, such as immune checkpoint blockade (ICB) and adoptive lymphocyte transfer (ALT), have garnered excitement over the past couple of decades due to their success in certain subsets of cancers. Broadening the repertoire of cancers in which immunotherapies are efficacious remains a major goal of cancer immunology groups worldwide.

During the past decade, chimeric antigen receptor (CAR) T cell therapy has emerged as a successful treatment for various cancers of the blood. This therapeutic strategy involves the ex vivo engineering of T cells to selectively recognize antigenic molecules expressed on the surface of tumor cells, resulting in immune system activation, tumor cell clearance, and improved patient outcomes. Although widely successful for the treatment of blood cancers, such as leukemias and lymphomas, CAR-T cell efficacy has remained limited for the treatment of solid tumors. At the root of failure in many solid cancers is substantial intratumoral heterogeneity. Many cancers possess few tumor-specific antigens and exhibit neoantigen expression profiles that vary even cell to cell.

Glioblastoma (GBM) is the most aggressive primary brain cancer with a median survival of less than 16 months and is one of the most lethal malignancies in humans. This dire prognosis signifies the urgent need for improved treatment options, such as immunotherapy. One hindrance to immunotherapy in GBM is the requirement for immune cells to be trafficked to the site of the tumor and survive the hostile immune suppressive tumor microenvironment. The other major hindrance to immunotherapy is the notoriously heterogeneous nature of GBM tumors. This intratumoral heterogeneity results in escape by antigen-negative tumor cell populations following CAR treatment. For example, the most prevalent GBM tumor-specific protein antigen, Epidermal Growth Factor Receptor (EGFR) vIII, is present in just 30% of tumors, and then on only 30-50% of cells. Hence, what is needed are novel means for identifying and targeting tumor cells that think “outside the box” and rely on more universally expressed targets to bypass the challenges conferred by marked tumor heterogeneity.

### BRIEF SUMMARY OF THE INVENTION

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

Glioblastoma (GBM) is a lethal malignancy with several barriers to care, localization and tumor heterogeneity. The present disclosure addresses these shortcomings by providing a novel chimeric antigen receptor (CAR) T cell that simultaneously targets wildtype EGFR (EGFRwt) and a mutant variant (EGFRvIII) with direct administration.

Accordingly, one aspect of the present disclosure provides a chimeric antigen receptor (CAR) comprising an extracellular domain comprising an antigen binding region which binds to both a wildtype EGFR and EGFR vIII, a transmembrane domain, and at least one intracellular domain comprising at least one co-stimulatory signaling domain.

The CAR described herein may comprise an extracellular antigen binding region with a single chain variable fragment of SEQ ID NO: 7 and SEQ ID NO: 9 or SEQ ID NO:12, comprising complementarity determining regions (CDR) of SEQ ID NO: 1-6. The single chain variable fragment heavy and the light chain may be connected by a linker.

In some embodiments, the CAR of the present disclosure further comprises a transmembrane domain and an intracellular domain comprising at least one co-stimulatory domain. Suitably, one co-stimulatory domain may be the CD3 zeta (CD3ζ) domain. In some embodiments, the co-stimulatory domains are selected from the group consisting of a signaling domain from CD28, 4-1BB, OX-40, ICOS, or other members of the TNF receptor or Ig superfamilies alone or in combination. In particular embodiments, the co-stimulatory signaling domains are selected from the group consisting of CD28, 4-1BB, CD3ζ, and combinations thereof.

In one embodiment, the CAR of the present disclosure further comprises a signal sequence linked to the extracellular domain.

In some embodiments, the CAR of the present disclosure the extracellular domain comprises the D2C7 single-chain variable fragment (ScFv), and the co-stimulatory domains comprise a CD28 co-stimulatory domain, a co-stimulatory signaling domain comprising 4-1BB, and a co-stimulatory signaling domain comprising CD3ζ.

Another aspect of the present disclosure provides a construct comprising a heterologous promoter operably connected to a polynucleotide encoding a CAR described herein. In particular, the construct may comprise a lentiviral, retroviral or AAV vector.

Another aspect of the present disclosure provides activated T cells expressing a CAR described herein, or a construct encoding a polynucleotide encoding a CAR described herein. In some embodiments, the CAR-T cells are present in a therapeutically effective amount for the prevention and/or treatment of a cancer that expresses wildtype EGFR (EGFRwt) and/or EGFRvIII. In some embodiments, the CAR-T cells are activated to produce one or more cytokines, accordingly, the cytokines may be selected from IL-1, IL-2, IL-4, IFN-γ, IL-10, IL-12, TNF-α and GM-CSF. A portion of the CAR-T cells of the present disclosure may be activated and express one or more markers including, CD2, CD28, CTLA4, CD40 ligand (gp39), CD18, CD25, CD69, CD16/CD56, MHC Class I, MHC Class II, CD8, CD4, CD3/TcR, CD54, LFA-1 and VLA-4. The CAR-T cells may further comprise a pharmaceutically acceptable excipient, carrier, and/or diluent which supports maintenance of the activated T cells.

Another aspect of the present disclosure provides a method of treating cancer in a subject suffering from an EGFR-associated cancer, the method comprising administering to the subject a therapeutically effective amount of a CAR-T cell described herein. Accordingly, the subject may be a human subject and the treatment may result in the induction of an anti-tumor response to the EGFR-associated cancer.

In some embodiments, the method of treating an EGFR-associated cancer may comprise intracranial or intrathecal administration of the CAR-T cells described herein.

In some embodiments, the cancer is located in the central nervous system and may be a glioma, glioblastoma, medulloblastoma, ependymoma or diffuse intrinsic pontine glioma (DIPG). In some embodiments, the cancer is a brain metastases or leptomeningeal disease.

Another aspect of the present disclosure is a method of preparing a population of activated T cells expressing a CAR comprising an extracellular domain which specifically binds EGFR and/or EGFR vIII variant, the method comprising: (i) contacting in vitro one or more T cells that have been modified to express the CAR with a stimulus that induces expansion of the T cells to provide an expanded T cell population; and (ii) activating in vitro the T cells to produce an activated T cell population. The activated T cell population may be administered to a subject to treat a cancer.

Another aspect of the present disclosure provides all that is described and illustrated herein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1A-1E. Schematic and graphs showing the generation of D2C7 CAR in accordance with one embodiment of the present disclosure. (A) Schematic of third generation human D2C7 CAR. (B) Mock, VIII, or D2C7 CAR were co-cultured with NR6, NR6 wt, NR6vIII, or no target cells at a 10:1 ratio. After 24 hours, supernatant was collected, diluted 1:10, and then analyzed for human Ifn-γ. (C-E) Mock, VIII, or D2C7 CAR were co-cultured at the indicated ratios with CellTrace Violet labeled NR6 wt, NR6vIII, or NR6 target cells. After 24 hours, surviving target cells were calculated by using flow cytometry and counting beads. % survival was calculated as follows: (#tumor cells/#target only cells)*100.

FIG. 2A-2B. D2C7 CAR potently kills the GBM line U87 and U87vIII in accordance with one embodiment of the present disclosure. Mock, VIII, or D2C7 CAR were co-cultured at the indicated ratios with CellTrace Violet labeled U87 and U87vIII. After 24 hours, surviving target cells were calculated by using flow cytometry and counting beads. % survival was calculated as follows: (#tumor cells/#target only cells)*100.

FIG. 3A-3B. D2C7 CAR prolongs survival in a homogeneous and heterogeneous in vivo model of glioma in accordance with one embodiment of the present disclosure. 25,000 U87vIII (A) or a mixture of 17,500 U87+7,500 U87vIII (B) cells were implanted intracranially in NOD scid gamma (NSG) mice. 48 hours later, 2×10⁶ Mock, VIII (A only), or D2C7 CARs were administered locally to the tumor site (intracranially). Mice were followed for survival.

FIG. 4A-4B. D2C7 CAR is effective against the DAOY medulloblastoma line. (A-B) DAOY cells derived from a 4 year old patient with an EGFRwt-expressing brain tumor. (A) In-vitro toxicity assay of DAOY cells treated with D2C7 or Mock CAR, (B) Survival of mice implanted intracranially with DAOY cells and treated with 2×10⁶. D2C7 or Mock CAR expressing T cells.

FIG. 5. D2C7 CAR is cytotoxic against the A-431 epidermoid carcinoma line, a potential model of brain metastasis. In-vitro cytotoxicity assay of EGFRwt expression human epidermoid carcinoma cells line, A-431, following treatment with D2C7 or Mock CAR expressing T cells.

### DETAILED DESCRIPTION OF THE INVENTION

Glioblastoma (GBM) is the most aggressive primary brain cancer with a median survival of less than 16 months. This dire prognosis necessitates the urgent need for improved treatment options, such as immunotherapy. T cells expressing chimeric antigen receptors (CAR) have helped revolutionize immunotherapy for hematological cancers but generally failed to control solid tumors. Challenges in immunotherapy for solid tumors include proper trafficking to the tumor site and tumor heterogeneity. The goal of the present work is to optimally develop a CAR to target solid tumors of the CNS.

The term “chimeric antigen receptor” or “chimeric receptor” or “CAR” or “CARs” as used herein refers to a polypeptide having a pre-defined binding specificity to a desired target and operably connected to (e.g., as a fusion or as separate chains linked by one or more disulfide bonds, etc.) the intracellular part of a T-cell activation domain. More particularly, CAR are engineered receptors, which, when expressed graft an antigen specificity onto a cytotoxic cell, for example T cells, NK cells or macrophages. Suitably, CAR proteins are engineered to give T cells the new ability to target a specific protein. The CARs of the present invention may comprise an extracellular domain with at least one antigen specific targeting region, a transmembrane domain (TM), and an intracellular domain (ID) including one or more co-stimulatory domains (CSD) in a combination that is not naturally found together on a single protein. This particularly includes receptors wherein the extracellular domain and the cytoplasmic domain are not naturally found together on a single receptor protein. Further, the chimeric receptor is different from the TCR expressed in the native T cell lymphocyte.

An extracellular domain is external to the cell or organelle and functions to recognize and respond to a ligand. A transmembrane domain spans the membrane of a cell, and an intracellular domain is situated inside a cell. Intracellular co-stimulatory domains provide secondary signals to the cell. They can recruit signaling molecules, cytoskeletal mobilization or induce cell proliferation, differentiation or survival. In the present disclosure a CAR may include an antigen specific extracellular domain, a transmembrane domain and one or more intracellular domains with one or more co-stimulatory domains.

The antigen binding domain of a CAR may bind to a single target, or multiple targets. The CAR of the present disclosure comprises an extracellular domain comprising an antigen binding region which binds to both wild type epidermal growth factor receptor (EGFR) and an EGFR vIII variant. EGFR is also known as ErbB-1, HER 1 and is a receptor that becomes activated by binding to specific ligands, including members of the EGF family of extracellular ligands. EGFR vIII is a variant of EGFR with an extracellular deletion mutation. EGFR vIII is expressed in some tumors, and considered a tumor specific antigen in glioblastoma tumors, despite being present in only 30% of tumors, and only approximately 30-50% of cells in the tumors.

The extracellular domain antigen binding region of the present disclosure comprises a single chain variable fragment (scFv) which is comprised of six complementarity determining regions. Complementary determining regions, or CDR, or CDRs, are hypervariable domains that determine specific antibody binding. In the present disclosure, the CDRs include SEQ ID NO: 1-6.

scFv are polypeptides that contain the variable light chain and variable heavy chain of an antibody connected by a flexible linker peptide. The scFv of the present disclosure may comprise the scFv of SEQ ID NO: 12 or sequences with at least 95% identity to SEQ ID NO: 12. The scFv of the present disclosure may also be the heavy chain variable region of SEQ ID NO: 7 and a light chain variable region of SEQ ID NO: 9 or sequences with at least 95% identity to SEQ ID NO: 7 and 9.

The linker may be 10-25 amino acids long and made up of glycine and serine amino acids, as well as optionally with dispersed hydrophilic residues to increase solubility. The linker keeps each of the variable regions at a distance that favors proper folding and formation of the antigen-binding site while also minimizing oligomerization of the scFv. A simple form of a linker is the hinge region of IgG1. The linker of the present disclosure may be a linker of SEQ ID NO: 8 or sequences with at least 95% identity to SEQ ID NO: 8.

The CAR of the present disclosure may comprise a transmembrane and hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety (e.g. EGFR/EGFRvIII scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11 a, CD18), ICOS (CD278), 4-1 BB (CD137), GITR, CD40, BAFFR, HVEM (LIGHTR), SLAMF7, NKp80 (KLRF1), CD160, CD19, IL2R beta, IL2R gamma, IL7R a, ITGA1, VLA1, CD49a, ITGA4, IA4, CD49D, ITGA6, VLA-6, CD49f, ITGAD, CD 11 d, ITGAE, CD103, ITGAL, CD11 a, LFA-1, ITGAM, CD11 b, ITGAX, CD11 c, ITGB1, CD29, ITGB2, CD18, LFA-1, ITGB7, TNFR2, DNAM 1 (CD226), SLAMF4 (CD244, 2B4), CD84, CD96 (Tactile), CEACAM 1, CRTAM, Ly9 (CD229), CD160 (BY55), PSGL1, CD 100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively, the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR. In some embodiments, the CAR has more than one transmembrane domain, which can be a repeat of the same transmembrane domain or can be different transmembrane domains. The CAR of the present disclosure may comprise a hinge and transmembrane domain of SEQ ID NO: 13 or sequences with at least 95% identity to SEQ ID NO: 13.

The CAR of the present disclosure may comprise at least one intracellular signaling domain, region or co-stimulatory molecule. The intracellular signaling domain may be a co-stimulatory domain. A costimulatory domain is required for an efficient antigen response in immune cells. In particular embodiments, the intracellular signaling domain is derived from CD3 zeta (CD3ζ (TCR zeta, GenBank accno. BAG36664.1). T-cell glycoprotein CD3 zeta (CD3ζ chain, also known as T-cell receptor T3 zeta chain or CD247 (Cluster of Differentiation 247), is a protein that in humans is encoded by the CD247 gene. The CAR of the present invention may also optionally comprise additional co-stimulatory domains, including CD28, 4-1BB, OX-40, ICOS or other members of the TNF receptor superfamily or immunoglobulin (Ig) superfamily. Members of the TNF superfamily form trimeric structures, and their monomers are composed of beta-strands that orient themselves into a two-sheet structure. The TNF superfamily ligands include lymphotoxin alpha, tumor necrosis factor, lymphotoxin beta, OX40 ligand, CD40 ligand, Fas ligand, CD27 ligand, CD30 ligand, CD137 ligand, TNF-related apoptosis-inducing ligand, receptor activator of nuclear factor kappa-B ligand, TNF-related weak inducer of apoptosis, a proliferation-inducing ligand, B-cell activating factor, LIGHT, vascular endothelial growth factor, TNF superfamily member 18 and ectodysplasin A. These ligands then bind to receptors in the TNF superfamily. Ig superfamily members are characterized based on shared structural features with immunoglobulins (aka antibodies), including an immunoglobulin domain with a characteristic Ig-fold. The Ig domain is reported to be one of the most populous family of proteins in the human genome with over 700 members identified and known in the art. Co-stimulatory domains of the present disclosure may comprise SEQ ID NO: 14, SEQ ID NO:15 or SEQ ID NO: 16 or sequences with at least 95% identity to SEQ ID NO: 14, SEQ ID NO:15 or SEQ ID NO: 16. These co-stimulatory domains may be used in isolation or in any combination.

While the CAR of the present disclosure is exemplified with the above mentioned co-stimulatory molecules, other co-stimulatory domains, including CD27, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2 can be used alone or in combination with other co-stimulatory molecules.

The CAR of the present disclosure may comprise a signal sequence or signaling domain. A signal sequence plays a determinant role in protein distribution and can allow the CAR to be glycosylated and anchored in the cell membrane. In some embodiments, the signal sequence comprises SEQ ID NO: 11 or a sequence having at least 95% identity to SEQ ID NO: 11.

By way of example, but not by way of limitation, in some embodiments, the disclosed CAR may comprise the scFv of D2C7 and the co-stimulatory domains comprise a CD28 co-stimulatory domain, a co-stimulatory signaling domain comprising 4-1BB, and a co-stimulatory signaling domain comprising CD3ζ. The CAR of the present disclosure may comprise SEQ ID NO.: 17 or SEQ ID NO: 18 (SS removed) or sequences having at least 95% identity to SEQ ID NOs: 17 or 18.

Some embodiments of the present disclosure describe a construct comprising a heterologous promoter operably connected to a polynucleotide encoding a CAR described herein. The term “construct” or “polynucleotide construct” is a polynucleotide which allows the encoded sequence to be replicated and/or expressed in the target cell. A construct may contain an exogenous promoter, operably linked to any one of the polynucleotides described herein. As used herein, a polynucleotide is “operably connected” or “operably linked” when it is placed into a functional relationship with a second polynucleotide sequence. As used herein, the terms “heterologous promoter,” “promoter,” “promoter region,” or “promoter sequence” refer generally to transcriptional regulatory regions of a gene, which may be found at the 5′ or 3′ side of a polynucleotides described herein, or within the coding region of said polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

The term “polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, including DNA, RNA, ORFs, analogs and fragments thereof.

In some embodiments, the construct is an expression construct, a vector or a viral vector. A vector is any particle used as a vehicle to artificially carry a foreign nucleic sequence, typically DNA, into another cell, where it can be replicated and/or expressed. A vector containing foreign DNA is termed recombinant DNA. The four major types of vectors are plasmids, viral vectors, cosmids, and artificial chromosomes. Expression constructs comprise a heterologous promoter and the nucleic acid sequence encoding protein of interest (e.g., EGFR/EGFR vIII) which is capable of expression in the cell in which it is introduced. The expression constructs include vectors which are capable of directing the expression of exogenous genes to which they are operatively linked. Such vectors are referred to herein as “recombinant constructs,” “expression constructs,” “recombinant expression vectors” (or simply, “expression vectors” or “vectors”) and may be used interchangeably. Suitable vectors are known in the art and contain the necessary elements in order for the gene encoded within the vector to be expressed as a protein in the host cell. The term “vector” refers to a nucleic acid molecule capable of transporting another nucleic acid to which it has been linked. One type of vector is a “plasmid”, which refers to a circular double stranded DNA loop into which additional DNA segments may be ligated, specifically exogenous DNA segments. Another type of vector is a viral vector, wherein additional DNA segments may be ligated into the viral genome. Viral vectors are incorporated into viral particles that are then used to transport the viral polynucleotide encoding the protein of interest into the target cells. Certain vectors are capable of autonomous replication in a host cell into which they are introduced. Other vectors can be integrated into the genome of a host cell upon introduction into the host cell, and thereby are replicated along with the host genome (e.g., lentiviral vectors). Moreover, certain vectors are capable of directing the expression of exogenous genes to which they are operatively linked. In general, vectors of utility in recombinant DNA techniques are often in the form of plasmids. In the present specification “vector” include expression vectors, such as viral vectors (e.g., replication defective retroviruses (including lentiviruses), adenoviruses and adeno-associated viruses (AAV)), which serve equivalent functions.

The vectors are heterogeneous exogenous constructs containing sequences from two or more different sources. Suitable vectors include, but are not limited to, plasmids, expression vectors, lentiviruses (lentiviral vectors), adeno-associated viral vectors (rAAV), among others and includes constructs that are able to express the protein of interest. A preferred vector is a lentiviral vector, retroviral vector or adeno-associated vector. Suitable methods of making viral particles are known in the art to be able to transform cells in order to express the protein of interest described herein.

Heterologous promoters useful in the practice of the present invention include, but are not limited to, constitutive, inducible, temporally-regulated, developmentally regulated, chemically regulated, tissue-preferred, tissue-specific promoters and cell-type specific. The heterologous promoter may be a plant, animal, bacterial, fungal, or synthetic promoter. Suitable promoters are known and described in the art. In mammalian cells, typical promoters include, without limitation, promoters for Rous sarcoma virus (RSV), human immunodeficiency virus (HIV-1), cytomegalovirus (CMV), SV40 virus, as well as the translational elongation factor EF-1α promoter or ubiquitin promoter.

Some embodiments of the present disclosure describe a CAR-T cell comprising activated T cells expressing a CAR described herein. In particular embodiments, the CAR-T cell comprises a construct encoding a CAR described herein. The term “CAR-T cells” as used herein refer to a T cell or population thereof, which has been modified through molecular biological methods to express a chimeric antigen receptor (CAR) on the T cell surface. T cells or T lymphocytes can be distinguished from other lymphocytes, such as B cells and natural killer cells (NK cells), by the presence of a T-cell receptor (TCR) on the cell surface. There are several subsets of T cells, each with a distinct function including helper T cells and cytotoxic T cells. T cells become activated when they are presented with peptide antigens. In some embodiments, some portion of activated T cells may express CD2, CD28, CTLA4, CD40 ligand (gp39), CD18, CD25, CD69, CD16/CD56, MHC Class I, MHC Class II, CD8, CD4, CD3/TcR, CD54, LFA-1 and VLA-4 on their cell surface, alone or in combination. In some cases, CAR engineered T cells of both CD8⁺ and CD4⁺ subsets can be recruited for redirected target cell recognition, bypass MHC class I and class II restriction.

Once activated, T cells divide rapidly and secrete small proteins called cytokines that regulate or assist in the active immune response. In some embodiments, the activated T cells may produce cytokines, including IL-1, IL-2, IL-4, IFN-γ, IL-10, IL-12, TNF-α and GM-CSF. Activated T cells can induce an immune response. The response can comprise, without limitation, specific immune response, non-specific immune response, both specific and non-specific response, innate response, primary immune response, adaptive immunity, secondary immune response, memory immune response, immune cell activation, immune cell proliferation, immune cell differentiation, and cytokine expression. The immune response may be specific to the T cell specific antigens including to EGFR and or EGFR vIII. In some embodiments, the immune response is an anti-tumor immune response.

In some embodiments of the present disclosure, activated CAR-T cells are present in a therapeutically effective amount for the prevention and/or treatment of a cancer. The anti-tumor immune response elicited by the disclosed CAR-modified T cells may be an active or a passive immune response. In addition, the CAR-mediated immune response may be part of an adoptive immunotherapy approach in which CAR-modified immune effector cells induce an immune response specific to EGFR or EGFR vIII. In some embodiment, the activated T cells are present in a therapeutically effective amount for the prevention and/or treatment of a cancer that expresses wildtype EGFR (EGFRwt) and/or the vIII variant (EGFRvIII). Effective amounts of CAR T cells can be determined by a physician with consideration of individual differences in age, weight, tumor size, extent of infection or metastasis, and condition of the patient (subject).

As used herein, a “therapeutically effective”, “an immunologically effective amount”, “an anti-tumor effective amount”, “a tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10¹⁰ cells/kg body weight, including all integer values within those ranges. In particular, the CAR described herein may be administered at a dose of about 2×10⁸ to 3×10⁹ cells per subject. T cell compositions may be administered once or administered multiple times. The cells can be administered by using infusion techniques that are commonly known in immunotherapy (see, e.g., Rosenberg et al., New Eng. J. of Med. 319: 1676, 1988). The optimal dosage and treatment regime for a particular patient can readily be determined by one skilled in the art of medicine by monitoring the patient for signs of disease and adjusting the treatment accordingly.

In some embodiments, the CAR-T cells may further comprise a pharmaceutically acceptable excipient, carrier, and/or diluent which supports maintenance of the activated T cells. As used herein, the term “carrier” refers to a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, may be found in the U.S. Pharmacopeia National Formulary, 1857-1859, (1990).

Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservative.

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

The present formulation may also comprise other suitable agents such as a stabilizing delivery vehicle, carrier, support or complex-forming species. Additionally, biologically acceptable buffer to maintain a pH close to neutral (e.g., 7.0-7.3). The coordinate administration methods and combinatorial formulations of the instant invention may optionally incorporate effective carriers, processing agents, or delivery vehicles, to provide improved formulations for delivery of the CAR described herein.

The CAR disclosed herein may also be co-administered with additional therapies. Co-administration refers to administration at the same time in an individual, and also may include administrations that are spaced by hours, days, weeks, or longer, as long as the administration of multiple therapeutic agents is the result of a single treatment plan. By way of example, the CAR-T effector cells of the present disclosure may be co-administered with other CAR-T cells having the same chimeric receptor and different co-stimulatory signaling domains and/or CAR-T cells that have a different chimeric receptor or with one or more additional therapeutic agents. Such agents may include, but are not limited to, chemotherapeutic agents, radiation, checkpoint inhibitor therapy, small molecule drugs, monoclonal antibodies, antibody-drug conjugates, and the like.

The co-administration may comprise administering the CAR-T effector cells of the present disclosure before, after, or at the same time as the alternative CAR-T cells and/or one or more additional therapeutic agents. In an exemplary treatment schedule, the CAR-T effector cells of the present disclosure may be given as an initial dose in a multi-day protocol, with alternative CAR-T cells and/or one or more additional therapeutic agents given on later administration days; or the alternative CAR-T cells and/or one or more additional therapeutic agents given as an initial dose in a multi-day protocol, with the CAR-T effector cells of the present disclosure given on later administration days. On another hand, alternative CAR-T cells and/or one or more additional therapeutic agents and the CAR-T effector cells of the present disclosure may be administered on alternate days in a multi-day protocol. In still another example, a mixture of alternative CAR-T cells and/or one or more additional therapeutic agents and the CAR-T effector cells of the present disclosure may be administered to reduce the number of proliferation-competent cells in a single administration while maintaining an effective T cell dose. This is not meant to be a limiting list of possible administration protocols.

### Methods

The present disclosure further provides a method of treating cancer in a subject suffering from an EGFR-associated cancer, the method comprising administering to the subject a therapeutically effective amount of a CAR-T cell to treat the cancer. EGFR may be found at high levels on some types of cancer cells, which causes these cells to grow and divide. EGFR-associated cancer are any cancers in which EGFR is present in an amount or location which deviates from typical patterns. Additionally, an EGFR-associated cancer may be a cancer with a mutated EGFR such that the function, expression or localization of EGFR is altered.

In some embodiments, the method comprises a treatment of the cancer which results in the induction of an anti-tumor response to the EGFR-associated cancer. Treating cancer in a subject includes the reducing, repressing, delaying or preventing cancer growth, reduction of tumor volume, and/or preventing, repressing, delaying or reducing metastasis of the tumor. Treating cancer in a subject also includes the reduction of the number of tumor cells within the subject. The term “treatment” can be characterized by at least one of the following: (a) reducing, slowing or inhibiting growth of cancer and cancer cells, including slowing or inhibiting the growth of metastatic cancer cells; (b) preventing further growth of tumors; (c) reducing or preventing metastasis of cancer cells within a subject; and (d) reducing or ameliorating at least one symptom of cancer. In some embodiments, an anti-tumor response may be an innate or adaptive immune response which leads to tumor control including a reduction in tumor volume, tumor growth, or tumor metastasis.

The term “subject” may be used interchangeably with the terms “individual” and “patient” and includes human and non-human mammalian subjects. In some embodiments, the subject is human. A subject as utilized herein may refer to a subject in need of treatment for a disease or disorder associated with a suspected tumor or cancer. A subject in need thereof may include a subject having a cancer that is characterized by gross abnormality visible by X-ray, computerized tomography (CT), or magnetic resonance imaging (MRI).

Some embodiments of the present disclosure provide a method of treating cancer comprising administering a therapeutically effective amount of a CAR, wherein the administration is intracranial or intrathecal. These routes of administration have the advantage of proper trafficking of CAR directly to the CNS and avoiding leakage of the CAR into the periphery. Intracranial injection is a direct method for drug delivery to the target site. In this method, intermittent bolus injections are administered locally in the brain, where the drugs are diffused with minimal systemic exposure and toxicity. Intracranial injections of the CAR may occur after surgery, for example in a resection cavity. Intrathecal administration is a route of administration for drugs via an injection into the spinal canal, or into the subarachnoid space so that it reaches the cerebrospinal fluid. Intrathecal administration has the advantage of avoiding being blocked by the blood brain barrier. Intrathecal administration may be administered into the ventricle of the brain. Intracranial injection or intrathecal injection may be used as a secondary therapy after, for example, radiation, chemotherapy or surgery to complement or augment standard of care. Additionally, the disclosed CAR-T cells may be administered either alone, or as a pharmaceutical composition in combination with diluents and/or with other components such as IL-2, IL-15, or other cytokines or cell populations or checkpoint inhibitors.

Some embodiments of the present disclosure provide methods for treating cancer with a therapeutically effective amount of a CAR described herein. In particular, the cancer may be located in the central nervous system including the brain and spinal cord. Suitably, the cancer may be a glioma, glioblastoma, medulloblastoma, ependymoma or diffuse intrinsic pontine glioma (DIPG). The cancer may also be a cancer that has metastasized to brain, or cerebrospinal fluid, spinal cord and/or meninges. These include, but not limited to brain metastases, Leptomeningeal disease, neoplastic meningitis, carcinomatous meningitis, lymphomatous meningitis, and leukemic meningitis. Cancers that commonly cause leptomeningeal disease include breast cancer, lung cancer, melanoma, acute lymphocytic leukemia (ALL) and Non-Hodgkin's lymphoma (NHL).

Some embodiments of the present disclosure provide methods of preparing a population of activated T cells expressing a CAR, the CAR comprising an extracellular domain which specifically binds EGFR and/or EGFR vIII variant, the method comprising: (i) contacting in vitro one or more T cells that have been modified to express the CAR with a stimulus that induces expansion of the T cells to provide an expanded T cell population; and (ii) activating in vitro the T cells to produce an activated T cell population.

The activated T cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan.

Following the collection of a patient's immune effector cells, the cells may be genetically engineered to express the disclosed CAR, via transduction, or any other means suitable to introduce the CAR into the T cell and infused back into the patient. Whether prior to or after genetic modification of the cells to express the CAR described herein, the cells can be activated and expanded in number using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and 20,060,121005. For example, the T cells of the invention may be expanded by contact with a surface having attached thereto an agent that stimulates a CD3/TCR complex associated signal and a ligand that stimulates a co-stimulatory molecule on the surface of the T cells. In particular, T cell populations may be stimulated by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD28 antibody include 9.3, B-T3, XR-CD28 (Diaclone, Besancon, France) and these can be used in the invention, as can other methods commonly known in the art (Berg et al., Transplant Proc. 30(8):3975-3977, 1998; Haanen et al., /. Exp. Med. 190(9): 13191328, 1999; Garland et al., /. Immunol. Meth. 227(1-2):53-63, 1999).

Where there is more than one administration in the present methods, the administrations can be spaced by time intervals of time, including minutes, hours, days, weeks, months or years. The dosing schedules encompass dosing for a total period of time. The invention is not limited to dosing intervals that are spaced equally in time, but encompass doses at non-equal intervals, such as a schedule consisting of administration at 1 day, 4 days, 7 days, and 25 days, to provide a non-limiting example. Dosing may occur in cycles or dosing schedules and an interval of non-dosing can occur between a cycle. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

### Additional Definitions

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

As used herein, the term “chimeric molecule” refers to a single molecule created by joining two or more molecules that exist separately in their native state. The single, chimeric molecule has the desired functionality of all of its constituent molecules. One type of chimeric molecules is a fusion protein.

As used herein, “identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

The term “specifically binds”, as used herein, when referring to a polypeptide (including antibodies) or receptor, refers to a binding reaction which is determinative of the presence of the protein or polypeptide or receptor in a heterogeneous population of proteins and other biologies. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10 s M⁻¹ (e.g., 10⁶ M-\ 10⁷ M-\ 10⁸ M-\ 10⁹ M-\ 10¹⁰ M″¹, 10¹¹ M″¹, and 10¹² M″¹ or more) with that second molecule.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

As used herein, the terms “prevent,” “preventing,” “prevention,” “prophylactic treatment” and the like refer to reducing the probability of developing a disease, disorder or condition in a subject, who does not have, but is at risk of or susceptible to developing a disease, disorder or condition.

The term “effective amount” or “therapeutically effective amount” refers to an amount sufficient to effect beneficial or desirable biological and/or clinical results.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. In one embodiment, the biological sample is a biopsy (such as a tumor biopsy). A biological sample may be obtained directly from a subject (e.g., by blood or tissue sampling) or from a third party (e.g., received from an intermediary, such as a healthcare provider or lab technician).

The term “disease” as used herein includes, but is not limited to, any abnormal condition and/or disorder of a structure or a function that affects a part of an organism. It may be caused by an external factor, such as an infectious disease, or by internal dysfunctions, such as cancer, cancer metastasis, and the like.

As is known in the art, a cancer is generally considered as uncontrolled cell growth. The methods of the present disclosure can be used to treat any cancer, and any metastases thereof, including, but not limited to, carcinoma, lymphoma, blastoma, sarcoma, and leukemia. In some embodiments, the cancer is characterized by, or associated with EGFR and/or the EGFR vIII variant (herein referred to as an “EGFR-associated cancer”). Suitable examples include, but are not limited to, of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma.

As used herein, the term “specifically” or “selectively” binds, when referring to a ligand/receptor, nucleic acid/complementary nucleic acid, antibody/antigen, or other binding pair (e.g., a cytokine to a cytokine receptor) indicates a binding reaction which is determinative of the presence of the protein in a heterogeneous population of proteins and other biologics. Thus, under designated conditions, a specified ligand binds to a particular receptor and does not bind in a significant amount to other proteins present in the sample. Specific binding can also mean, e.g., that the binding compound, nucleic acid ligand, antibody, or binding composition derived from the antigen-binding site of an antibody, of the contemplated method binds to its target with an affinity that is often at least 25% greater, more often at least 50% greater, most often at least 100% (2-fold) greater, normally at least ten times greater, more normally at least 20-times greater, and most normally at least 100-times greater than the affinity with any other binding compound.

“Administration” as it applies to a human, primate, mammal, mammalian subject, animal, veterinary subject, placebo subject, research subject, experimental subject, cell, tissue, organ, or biological fluid, refers without limitation to contact of an exogenous ligand, reagent, placebo, small molecule, pharmaceutical agent, therapeutic agent, diagnostic agent, or composition to the subject, cell, tissue, organ, or biological fluid, and the like. “Administration” can refer, e.g., to therapeutic, pharmacokinetic, diagnostic, research, placebo, and experimental methods. Treatment of a cell encompasses contact of a reagent to the cell, as well as contact of a reagent to a fluid, where the fluid is in contact with the cell. “Administration” also encompasses in vitro and ex vivo treatments, e.g., of a cell, by a reagent, diagnostic, binding composition, or by another cell.

“Attenuation” and “attenuated” encompasses a bacterium, virus, parasite, infectious organism, prion, cell (e.g., tumor cell, T cell), gene in the infectious organism, and the like, that is modified to reduce toxicity to a host. The host can be a human or animal host, or an organ, tissue, or cell. The bacterium, to give a non-limiting example, can be attenuated to reduce binding to a host cell, to reduce spread from one host cell to another host cell, to reduce extracellular growth, or to reduce intracellular growth in a host cell. Attenuation can be assessed by measuring, e.g., an indicum or indicia of toxicity, the LD₅₀, the rate of clearance from an organ, or the competitive index. Generally, an attenuation results an increase in the LD₅₀ and/or an increase in the rate of clearance by at least 25%; more generally by at least 50%; most generally by at least 100% (2-fold); normally by at least 5-fold; more normally by at least 10-fold; most normally by at least 50-fold; often by at least 100-fold; more often by at least 500-fold; and most often by at least 1000-fold; usually by at least 5000-fold; more usually by at least 10,000-fold; and most usually by at least 50,000-fold; and most often by at least 100,000-fold.

Other aspects of the present disclosure provide all that is described and illustrated herein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The following Examples are provided by way of illustration and not by way of limitation.

### EXAMPLES

### Example 1. Generation of a Third Generation CAR T Cell that Simultaneously Targets Wildtype EGFR and EGFRvIII for Treatment of Glioblastoma

Glioblastoma (GBM) is the most aggressive primary brain cancer with a median survival of less than 16 months. This dire prognosis signifies the urgent need for improved treatment options, such as immunotherapy. Chimeric antigen receptor (CAR) T cells have helped revolutionize immunotherapy, achieving considerable success in eliminating hematological cancers but generally failing to control solid tumors. One challenge to immunotherapy for GBM is proper trafficking into the CNS. The other major hindrance to CAR T cell success in solid tumors is tumor heterogeneity. Tumor-associated or tumor-specific antigens (TAA or TSA, respectively) are rarely expressed by all malignant cells within a tumor.

GBM is a particularly heterogenous cancer. As a specific example in GBM, the most prevalent TSA, EGFRvIII, is present in just 30% of tumors, and then on only 30-50% of cells. Therefore immunotherapy for GBM requires a novel targeting strategy that accounts for proper immune trafficking in the CNS and tumor heterogeneity.

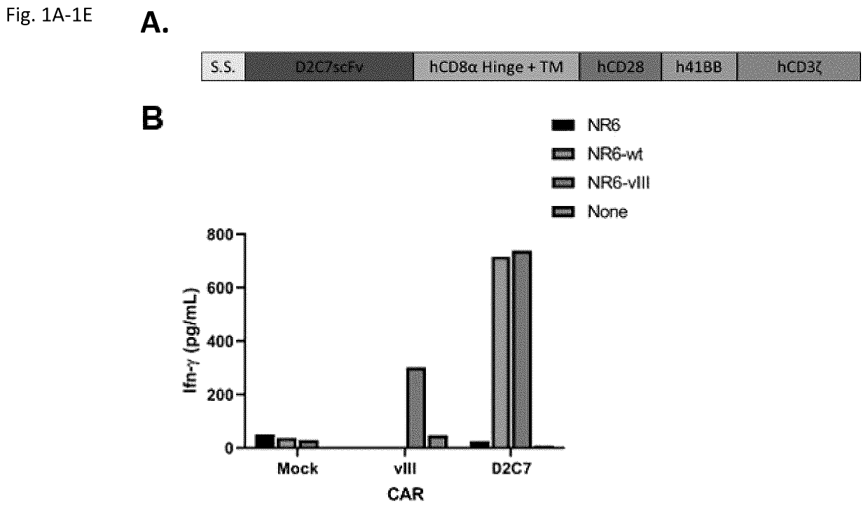

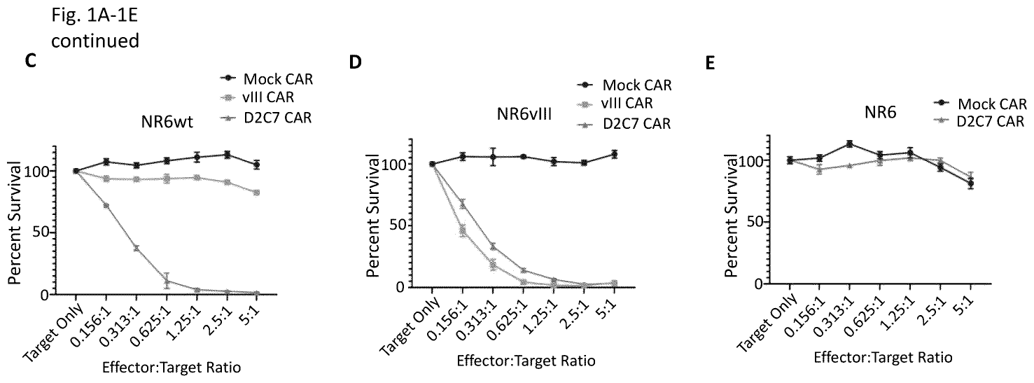

To validate the strategy of developing a chimeric antigen receptor (CAR) T cell that simultaneously targets wildtype EGFR (EGFRwt) and the vIII variant (EGFRvIII), activated human CD3 T cells were transduced with a retroviral construct encoding the D2C7 single-chain variable fragment (scFv) in tandem with the signaling domains of human CD28, 4-1BB, and CD3ζ to generate the third generation D2C7 CAR (FIG. 1A). D2C7 CAR specificity and cytotoxic function were initially evaluated by co-culturing D2C7 CAR with a murine fibroblast line (NR6) transfected to express human EGFRwt (NR6 wt) or human EGFRvIII (NR6vIII). After 24 hours of co-culture with NR6, NR6 wt, or NR6vIII, CAR-mediated interferon-γ (IFN-γ) release was measured by enzyme-linked immunosorbent assay (ELISA) (FIG. 1B). D2C7 CAR secreted IFN-γ in response to challenge with NR6 wt and NR6vIII; whereas, an EGFRvIII-specific (VIII) CAR only released IFN-γ in response to NR6vIII, while a mock-transduced CAR failed to release IFN-γ under any condition. Neither CAR released IFN-γ when co-cultured with NR6.

Direct cell cytotoxicity was analyzed by co-culturing D2C7, VIII, or Mock CAR with fluorescently-labeled NR6 wt, NR6vIII, or NR6 cells at various CAR (effector) to target ratios (FIG. 1C-E). After 24 hours of co-culture, remaining viable target cells were enumerated by flow cytometry and normalized to “target only” samples to generate a percent survival. D2C7 CAR potently killed NR6 wt and NR6vIII, with near total tumor cytotoxicity at less than 1:1 effector:target ratio. D2C7 and VIII CAR showed comparable target cell killing when challenged with NR6vIII. Importantly, D2C7 CAR did not kill EGFR-negative NR6 cells. Together, these data provide evidence that D2C7 specifically targets and kills EGFRwt and EGFRvIII-expressing target cells.

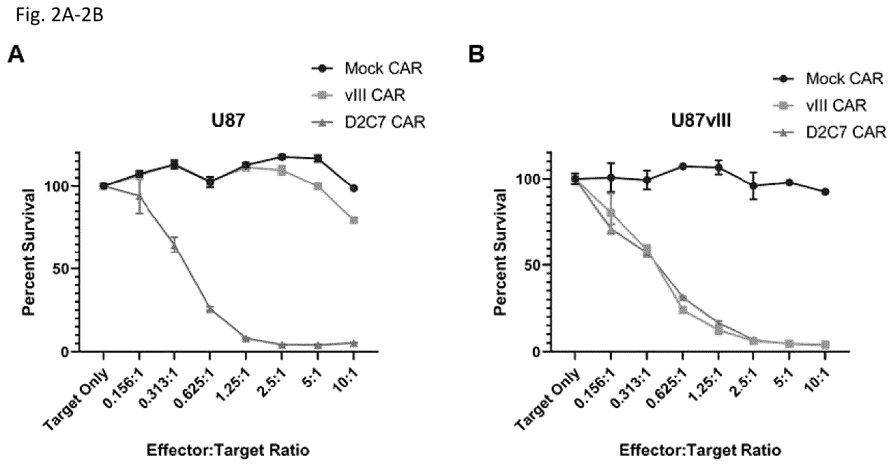

Subsequently, D2C7 CAR cytotoxicity was measured against the human-derived glioblastoma line U87 and U87 transfected with EGFRvIII (U87vIII) (FIGS. 2A and B). Again, D2C7 CAR displayed potent cytotoxicity against both U87 and U87vIII; whereas, VIII CAR only showed cytotoxicity against U87vIII. These data provide further evidence that D2C7 is specific and effective in killing a relevant, CNS-derived tumor cell line.

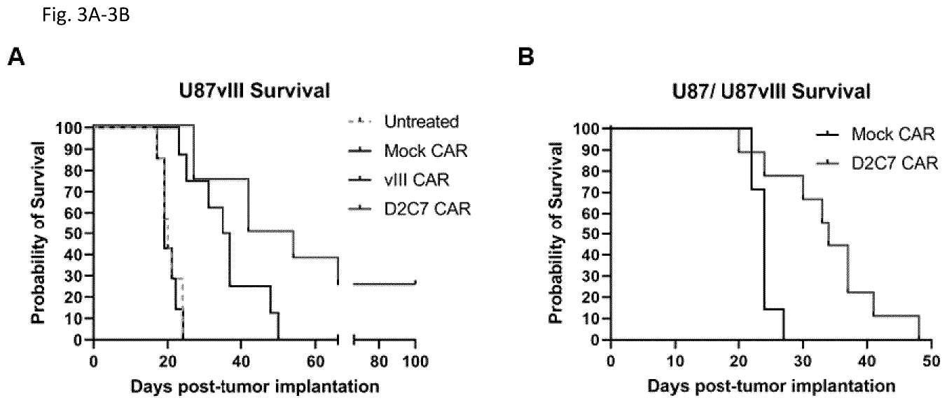

Next, it was sought to determine in vivo efficacy of D2C7 CAR. NOD scid gamma (NSG) mice were implanted intracranially with U87vIII (FIG. 3A) or a 70:30 mixture of U87/U87vIII (FIG. 3B). 48 hours later, 2×10⁶ Mock CARs, D2C7 CARs, or VIII CARs (in 3A) were administered intracranially, or mice were left untreated. In a homogeneous U87vIII tumor model, mice receiving D2C7 CARs had a median survival of 48 days compared to 19 days in mice receiving Mock CARs. Most interestingly, D2C7 CAR also outperformed the VIII CAR in this model. D2C7 CAR administration also resulted in 25% long-term survival compared to no long-term survival in the other groups. These unexpected results demonstrate the effectiveness of a CAR that simultaneously targets EGFR/EGFRVIII, even in a homogenous vIII tumor model. Importantly, D2C7 CAR significantly prolonged survival in a heterogeneous U87/U87vIII tumor model (FIG. 3B).

### Example 2. EGFR/EGFRVIII Targeted CAR-T Cells in Medulloblastoma and Central Nervous System Metastasis

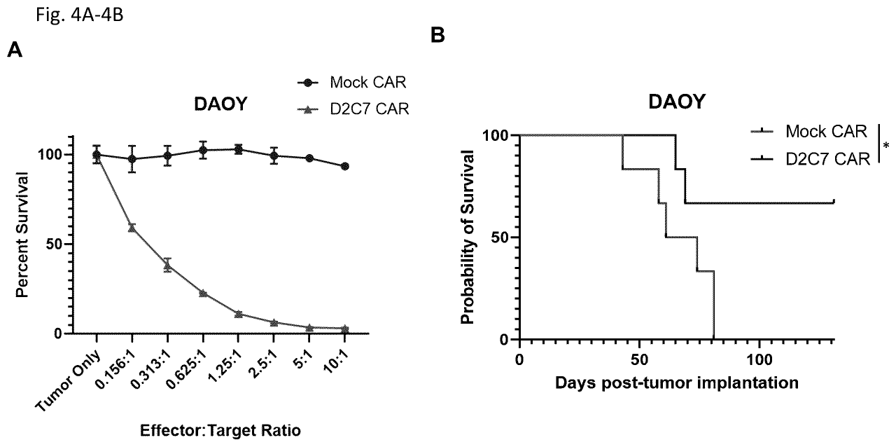

To determine if D2C7 CAR is effective beyond GBM, we challenged D2C7 CAR against a medulloblastoma cell line: DAOY. DAOY cells were derived from a 4-year old patient and represents a model of EGFRwt-expressing pediatric brain tumor. In FIG. 4A, we performed an in vitro cytotoxicity assay which showed potent killing of DAOY cells by D2C7 CAR. We next implanted DAOY cells IC in NSG mice followed by treatment with 2×10⁶ D2C7 or Mock CAR intracranially injected and monitored survival. D2C7 CAR was highly effective, resulting in 66% long-term survivors (FIG. 4B).

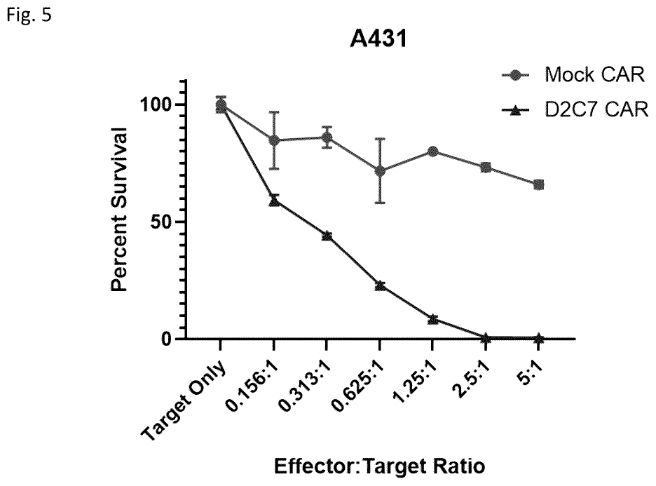

Another potential application for D2C7 CAR is for EGFRwt and/or EGFRvIII-expressing tumors that have metastasized to the central nervous system. Therefore, we tested D2C7 CAR against the EGFRwt-expressing human epidermoid carcinoma cell line A-431. An in vitro cytotoxicity assay suggests D2C7 CAR effectively kills A-431 cells (FIG. 5).

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A chimeric antigen receptor (CAR) comprising an extracellular domain comprising an antigen binding region which binds to both a wildtype EGFR and an EGFR vIII variant, a transmembrane domain, and at least one intracellular domain comprising at least one co-stimulatory signaling domain, wherein the antigen binding region comprises a single chain variable fragment (scFv) and the scFv comprises complementarity determining regions (CDR) of SEQ ID NOs: 1-6.

2. (canceled)

3. The CAR of claim 1, wherein the antigen binding region includes an scFv comprising a heavy chain variable region of SEQ ID NO: 7 and a light chain variable region of SEQ ID NO: 9 or sequences with at least 95% identity to SEQ ID NO: 7 and 9.

4. (canceled)

5. The CAR of claim 1, wherein the extracellular domain comprises the single-chain variable fragment (ScFv) of SEQ ID NO: 12 or sequences with at least 95% identity to SEQ ID NO: 12.

6. The CAR of claim 1, wherein the transmembrane domain comprises SEQ ID NO: 13 or sequences with at least 95% identity to SEQ ID NO: 13 and wherein the at least one co-stimulatory signaling domain comprises a CD3 zeta domain comprising SEQ ID NO: 16 or a sequence with at least 95% identity to SEQ ID NO: 16.

7. (canceled)

8. (canceled)

9. The CAR of claim 1, wherein the co-stimulatory signaling domains are selected from the group consisting of a signaling domain from CD28, 4-1BB, OX-40, ICOS, CD3 zeta and members of the TNF receptor superfamily or Ig superfamily.

10. (canceled)

11. The CAR of claim 1, wherein the co-stimulatory signaling domains comprises at least one of SEQ ID NOs: 14-16 or sequences having at least 95% identity to sequences 14-16.

12. (canceled)

13. The CAR of claim 1, wherein the CAR further comprises a signal sequence linked to the extracellular domain comprising SEQ ID NO: 11 or sequences with at least 95% identity to SEQ ID NO: 11.

14. (canceled)

15. The CAR of claim 1, wherein the extracellular domain comprises the D2C7 single-chain variable fragment (ScFv), and the co-stimulatory domains comprise a CD28 co-stimulatory domain, a co-stimulatory signaling domain comprising 4-1BB, and a co-stimulatory signaling domain comprising CD3ζ.

16. The CAR of claim 1, wherein the CAR comprises SEQ ID NO.: 17, SEQ ID NO: 18 or sequences having at least 95% identity to SEQ ID NO: 17-18.

17. A construct comprising a heterologous promoter operably connected to a polynucleotide encoding the CAR of claim 1.

18. The construct of claim 17, wherein the construct comprises a lentiviral, retroviral or AAV vector.

19. A chimeric antigen receptor (CAR)-T cell comprising a T cell expressing the chimeric antigen receptor (CAR) of claim 1.

20. A CAR-T cell comprising the construct of claim 17.

21. (canceled)

22. The CAR-T cell of claim 19, wherein the T cell is activated and produces one or more cytokines.

23. (canceled)

24. (canceled)

25. A pharmaceutical composition comprising the CAR T cell of claim 19, and a pharmaceutically acceptable excipient, carrier, and/or diluent which supports maintenance of the T cells.

26. A method of treating cancer in a subject suffering from an EGFR-associated cancer, the method comprising administering to the subject a therapeutically effective amount of the CAR-T cell of claim 19 to treat the cancer, wherein treatment of the cancer results in induction of an anti-tumor response to the EGFR-associated cancer.

27. (canceled)

28. (canceled)

29. The method of claim 26, wherein administration is intracranial or intrathecal.

30. (canceled)

31. The method of claim 26, wherein the cancer is a glioma, glioblastoma, medulloblastoma, ependymoma or diffuse intrinsic pontine glioma (DIPG) a brain metastases or leptomeningeal disease.

32. (canceled)

33. A method of preparing a population of activated T cells expressing a chimeric antigen receptor (CAR), the CAR comprising an extracellular domain which specifically binds EGFR and/or EGFR vIII variant, a transmembrane domain, and at least one intracellular domain comprising at least one co-stimulatory signaling domain, the method comprising: (i) contacting in vitro one or more T cells that have been modified to express the CAR with a stimulus that induces expansion of the T cells to provide an expanded T cell population; and (ii) activating in vitro the expanded T cell population to produce an activated T cell population.

34. The method of claim 33, wherein the CAR comprises SEQ ID NO.: 17, SEQ ID NO: 18 or sequences having at least 95% identity to SEQ ID NO: 17-18.

35. (canceled)
